(19) United States
(12) Patent Application Publication
(10) Pub. No.: US 2026/0204393 A1
(43) Pub. Date: Jul. 16, 2026

(54) CLIENT DATA MANAGEMENT COMPUTING APPARATUS WITH MULTI-STEP DATA ANALYTICS ENGINE FOR GENERATING PLAN DATA

(71) Applicant: Catalight Foundation, Walnut Creek, CA (US)

(72) Inventors: Lindsey SNEED, San Bruno, CA (US); Kalina Hatzell, Fairfield, CA (US); Nina Rudnick, Berkeley, CA (US); Ramana Linnea Gasch, Santa Cruz, CA (US)

(21) Appl. No.: 19/542,373

(22) Filed: Feb. 17, 2026

(51) Int. Cl. G16H 20/70; G06Q 50/20

(52) U.S. Cl. CPC G16H 20/70; G06Q 50/205

(57) ABSTRACT

A computing apparatus includes a processor, a communication interface coupled via a bus configured to communicate over a communication network with provider computing devices, external data server devices, and clinical server devices, and a memory storing a decision engine, workflow engine, outcome analysis tool, parameter recommendation tool, curriculum rules database, and hours rules database. The processor receives electronic requests, retrieves client specific data from external data server devices, and analyzes the data using automated evaluation assessment tools. The decision engine correlates client specific data with prior stored client outcome data to identify matching data patterns, improving accuracy. The workflow engine automatically processes and correlates evaluation data with stored curriculum and hour rules from clinical server devices to determine parameters without manual intervention, reducing processing time. Plan data is generated by correlating scores with client data stored in memory and transmitted over the communication network, standardizing data processing across multiple provider computing devices.

This application is a continuation of U.S. patent application Ser. No. 16/530,632, filed Aug. 2, 2019, and which claims the benefit of U.S. Provisional Patent Application Ser. No. 62/714,525, filed Aug. 3, 2018, both of which are hereby incorporated by reference in their entirety.

### CROSS REFERENCE TO RELATED APPLICATIONS

This application is related to U.S. Provisional Patent Application Ser. No. 62/461,184 filed Feb. 20, 2017 entitled “System and Method for Managing Treatment Plans,” which is hereby incorporated by reference in its entirety.

### FIELD

This technology is generally directed to methods for applying advanced multi-step analytics to generate treatment plan data and devices thereof.

### BACKGROUND

As illustrated in FIG. 1, a typical mental healthcare cycle may consist of several sequential and independently performed steps including screening, referral, intake, placement, assessment, treatment, and discharge, from where the client can be re-referred to the screening step, as needed. In particular, during the assessment step, a client may be evaluated and then an assessment report may be produced to authorize a treatment strategy based on the manual assessment. The resulting assessment report can recommend a set of treatment parameters that include a curriculum and treatment hours.

Within this typical mental healthcare cycle, one type of tool which may be utilized is known as Applied Behavior Analysis (ABA). ABA is a scientific discipline that focuses on the analysis, design, implementation, and evaluation of social and other environmental modifications to produce meaningful changes in behavior. Based on standardized assessment and clinical observations ABA programs make changes to an individual's environment to promote changes to that person's behavior. ABA is based on the premise that an individual's behavior is determined by past and current environmental events in conjunction with genetic and physiological variables. When included in a client's treatment for autism spectrum disorder (ASD), ABA focuses on addressing the problems of the disorder by altering the individual's social and learning environments. The focus of ABA is both on skill acquisition (behaviors to increase) as well as behavior reduction for maladaptive behaviors. When occurring together, these can create meaningful change to an individual's social functioning.

Another type of tool which may be utilized is known as, Evidence-Based Practice (EBP). EBP is a process in which the practitioner combines well-researched interventions with clinical experience and ethics, and client preferences and culture to guide and inform the delivery of treatments and services. In order to achieve EBP, the practitioner, researcher, and client must work together in order to identify what works, for whom and under what conditions. This approach ensures that the treatment and services, when used as intended, will have the most effective outcomes as demonstrated by the research. EBP requires a system that enables standardization around how research guidelines can be implemented and how client data can be collected, in order to recommend quality treatment.

Unfortunately, this typical mental healthcare cycle has been inconsistent, inefficient, and has failed to effectively integrate available tools or to identify, process and utilize and cross correlate use of these tools and correlated stored data to improve treatment strategies. Prior software tools have primarily focused on just data recordation and reporting. These prior software tools have neither developed any effective analytics nor been unable to take advantage of the vast and available stored client outcome data to provide better and more consistent mental healthcare. This has been particularly problematic in certain areas, such as for example autism or other spectrum disorders, which can be particularly challenging to effectively treat because of the unique personal and situational factors impacting each client and the ongoing developments in best practices and research which are not effectively identified and utilized.

### SUMMARY

A method for applying advanced multi-step analytics to generate treatment plan data includes analyzing, by a computing device, in response to an electronic request client specific data associated with a client identifier based on one or more automated assessment tools to generate assessment data associated with the client identifier. One or more treatment parameters from a plurality of stored treatment parameters are determined, by the computing device, based on the client specific data, the assessment data, and one or more of the plurality of stored curriculum and hour rules. One or more curriculum based assessment scores are calculated, by the computing device, using one or more automated scoring assessment tools based on the determined one or more treatment parameters. Treatment plan data is generated, by the computing device, based on the curriculum based assessment scores and at least one of a plurality of other types of client data. The generated treatment plan data is provided, by the computing device, in response to the electronic request.

A non-transitory computer readable medium having stored thereon instructions comprising executable code which when executed by one or more processors, causes the one or more processors to analyze in response to an electronic request client specific data associated with a client identifier based on one or more automated assessment tools to generate assessment data associated with the client identifier. One or more treatment parameters from a plurality of stored treatment parameters are determined based on the client specific data, the assessment data, and one or more of the plurality of stored curriculum and hour rules. One or more curriculum based assessment scores are calculated using one or more automated scoring assessment tools based on the determined one or more treatment parameters. Treatment plan data is generated based on the curriculum based assessment scores and at least one of a plurality of other types of client data. The generated treatment plan data is provided in response to the electronic request.

A computing apparatus comprising a memory coupled to a processor which is configured to be capable of executing programmed instructions stored in the memory to analyze in response to an electronic request client specific data associated with a client identifier based on one or more automated assessment tools to generate assessment data associated with the client identifier. One or more treatment parameters from a plurality of stored treatment parameters are determined based on the client specific data, the assessment data, and one or more of the plurality of stored curriculum and hour rules. One or more curriculum based assessment scores are calculated using one or more automated scoring assessment tools based on the determined one or more treatment parameters. Treatment plan data is generated based on the curriculum based assessment scores and at least one of a plurality of other types of client data. The generated treatment plan data is provided in response to the electronic request.

This technology provides a number of advantages including providing methods, devices, and non-transitory computer readable media that apply advanced multi-step analytics to generate treatment plan data that is aligned with current best practices and research and effectively and consistently tailored to each client. The advanced multi-step analytics utilize client specific data including situational data and preference data for a client as well as other stored client outcome data correlated to other client identifiers with one or more matching types of diagnosis conditions. Additionally, these advanced multi-step analytics may utilize artificial intelligence (AI) to analyze data including the client specific data and the other stored client data including other client stored treatment plan data and corresponding treatment outcome data in order to generate and continually update executable rules for new assessments and adjustments to treatment parameters for generating mental healthcare treatment plan data. Further, these advanced multi-step analytics may include analytics to customize the duration of different parts of the treatment strategy. Accordingly, this technology generates much more effective and consistent treatment plan data and does so in substantially less time. Further, with this technology any changes to the particular healthcare team or between different healthcare providers associated with each client identifier no longer impacts the generated treatment plan data for each client identifier.

### BRIEF DESCRIPTION OF THE DRAWINGS

FIG. 1 is a diagram of an example of a prior typical care cycle;

FIG. 2 is a block diagram of an environment with an example of a treatment management computing apparatus;

FIG. 3 is a block diagram of the example of the treatment management computing apparatus shown in FIG. 2;

FIG. 4 is a flow chart of an example of a method for applying advanced multi-step analytics to generate treatment plan data associated with a client identifier for a client with Autism Spectrum Disorder (ASD);

FIG. 5 is a functional diagram of the example of applying advanced multi-step analytics to generate treatment plan data in FIG. 4;

FIG. 6 is a functional block diagram of another example of applying advanced multi-step analytics to generate treatment plan data in FIG. 4;

FIG. 7 is a diagram of an example of a curriculum+baseline hours rule table;

FIG. 8 is a diagram of an example additional hours table;

FIG. 9A is a screenshot of an example of a graphical user interface of an example of a clinical hours and assessment recommendation tool provided by the treatment management computing apparatus to a provider computing device;

FIG. 9B is a screenshot of an example of a graphical user interface of generated assessment data provided by the treatment management computing apparatus to a provider computing device; and

FIG. 9C is a screenshot of an example of a graphical user interface of treatment plan data comprising curricula recommendations, hours recommendations, and additional hours recommendations provided by the treatment management computing apparatus to a provider computing device.

### DETAILED DESCRIPTION

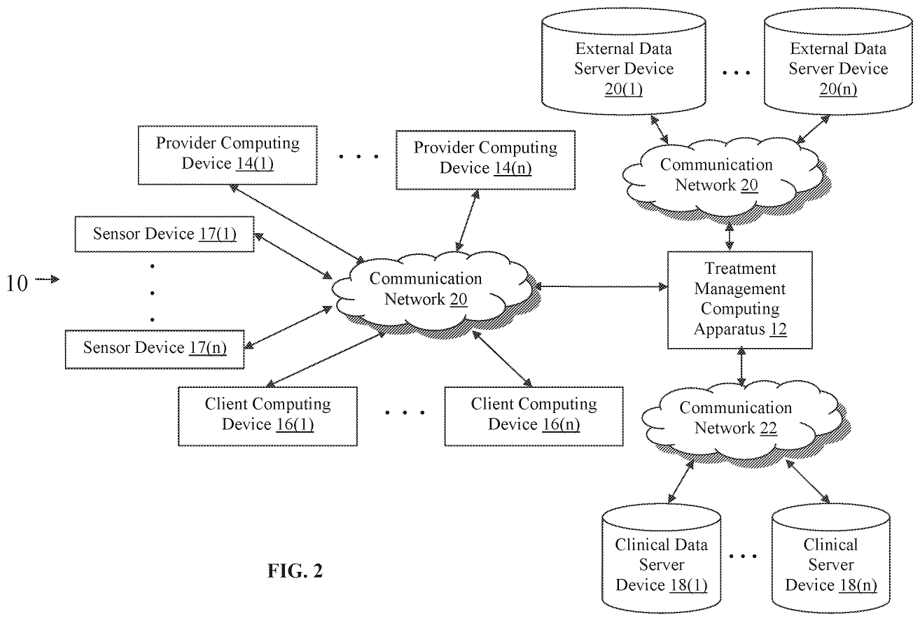

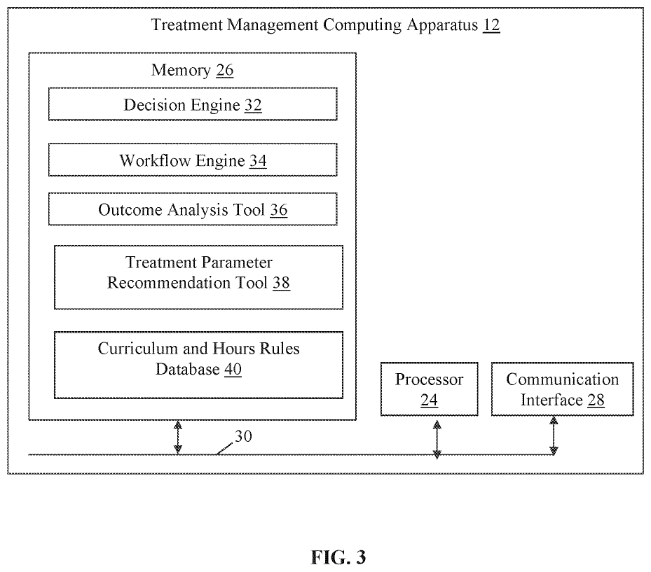

An environment 10 with an example of a treatment management computing apparatus 12 that applies advanced multi-step analytics to generate treatment plan data is illustrated in FIGS. 2-3. In this particular example, the environment 10 includes the treatment management computing apparatus 12, provider computing devices 14(1)-14(n), client computing devices 16(1)-16(n), client sensor devices 17(1)-17(n), clinical server devices 18(1)-18(n), and external data server devices 20(1)-20(n) coupled via one or more communication networks 22, although the environment could include other types and numbers of systems, devices, components, and/or other elements as is generally known in the art and will not be illustrated or described herein. This technology provides a number of advantages including providing methods, devices, and non-transitory computer readable media that apply advanced multi-step analytics to generate treatment plan data that is aligned with current best practices and research.

Referring more specifically to FIG. 3, the treatment management computing apparatus 12 may perform any number of functions and other operations as illustrated and described by way of the examples herein. The treatment management computing apparatus 12 may include one or more processor(s) 24, a memory 26, and a communication interface 28, which are coupled together by a bus or other communication link 30, although the treatment management computing apparatus 12 can include other types and/or numbers of elements in other configurations.

The processor(s) 24 of the treatment management computing apparatus 12 may execute programmed instructions stored in the memory of the treatment management computing apparatus 12 for any number of functions and other operations as described and illustrated by way of the examples herein. The processor(s) of the treatment management computing apparatus 12 may include one or more CPUs or general purpose processors with one or more processing cores, for example, although other types of processor(s) can also be used.

The memory 26 of the treatment management computing apparatus 12 may store these programmed instructions for one or more aspects of the present technology as described and illustrated herein, although some or all of the programmed instructions could be stored elsewhere. A variety of different types of memory storage devices, such as random access memory (RAM), read only memory (ROM), hard disk, solid state drives, flash memory, or other computer readable medium which is read from and written to by a magnetic, optical, or other reading and writing system that is coupled to the processor(s), can be used for the memory.

Accordingly, the memory 26 of the treatment management computing apparatus 12 can store one or more applications that can include computer executable instructions that, when executed by the treatment management computing apparatus 12, cause the treatment management computing apparatus 12 to perform actions, such as to transmit, receive, execute analytics or otherwise process data, for example, and to perform other actions as described and illustrated by way of the examples herein. The application(s) can be implemented as modules or components of other applications. Further, the application(s) can be implemented as operating system extensions, module, plugins, or the like. In this particular example, the memory 26 includes a decision engine 32, a workflow engine 34, an outcome analysis tool 36, a treatment parameter recommendation tool 38, an artificial intelligence engine 40, a curriculum rules database 42, and an hours rules database 44, although the memory 26 can comprise other types and/or numbers of other modules, programmed instructions and/or data.

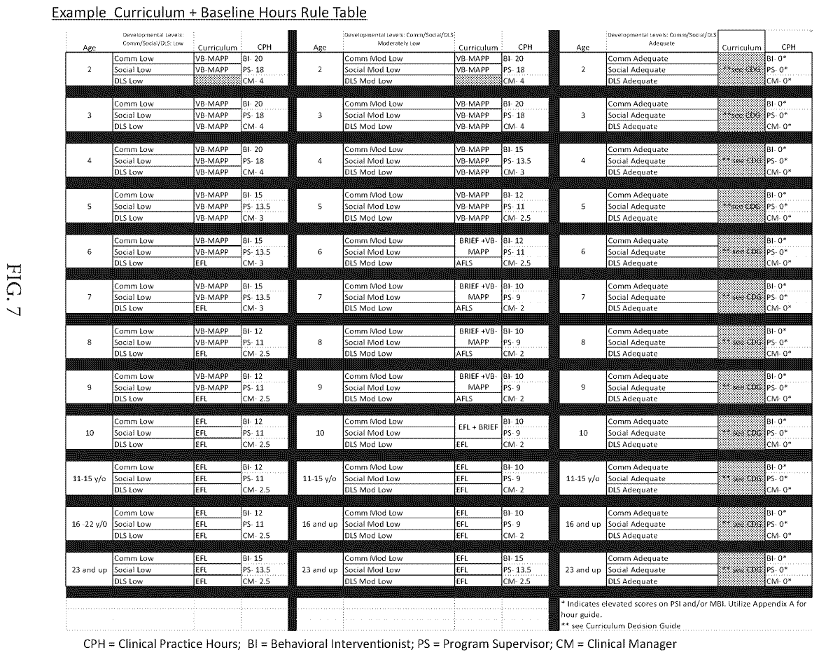

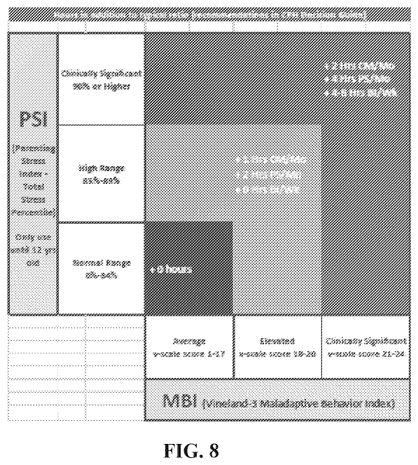

The decision engine 32 may include programmable instructions to generate treatment plan data by way of example only, although this engine may have other types and/or numbers of programmed instructions and/or data. By way of example, the decision engine may use artificial intelligence trained on prior outcome data correlated to similar types of diagnosed conditions as described and illustrated by way of examples herein. The workflow engine 34 may include programmable instructions to process prior outcome data and correlate portions to current client specific data which is provided to the decision engine 32 by way of example only, although this engine may have other types and/or numbers of programmed instructions and/or data. The outcome analysis tool 36 may include programmable instructions as well as rules and other data to process and manage outcome data against current best practices and research by way of example only, although this tool may have other types and/or numbers of programmed instructions and/or data. The treatment parameter recommendation tool 38 may include programmed instructions to identify one or more treatment parameters from stored treatment parameters associated with particular types of diagnosed conditions by way of example only, although this tool may have other types and/or numbers of programmed instructions and/or data. The curriculum and hours rules database 40 may include stored curriculum and associated hours as illustrated in FIGS. 7 and 8 by way of example only, although this database may other types and/or numbers of programmed instructions and/or data. Further examples of the programmed instructions and/or data in the decision engine 32, the workflow engine 34, the outcome analysis tool 36, the treatment parameter recommendation tool 38, and the curriculum and hours rules database 40 are illustrated and described by way of the examples herein.

Even further, the application(s) may be operative in a cloud-based computing environment. The application(s) can be executed within or as virtual machine(s) or virtual server(s) that may be managed in a cloud-based computing environment. Also, the application(s), and even the treatment management computing apparatus 12 itself, may be located in virtual server(s) running in a cloud-based computing environment rather than being tied to one or more specific physical network computing devices. Also, the application(s) may be running in one or more virtual machines (VMs) executing on the network traffic management apparatus. Additionally, in one or more embodiments of this technology, virtual machine(s) running on the treatment management computing apparatus 12 may be managed or supervised by a hypervisor.

The communication interface 28 of the treatment management computing apparatus 12 operatively couples and communicates between the treatment management computing apparatus 12 and other device and/or systems, such as provider computing devices 14(1)-14(n), client computing devices 16(1)-16(n), client sensor devices 17(1)-17(n), clinical server devices 18(1)-18(n), and external data server devices 20(1)-20(n), which are all coupled together by the communication network(s) 22, although other types and/or numbers of communication networks or systems with other types and/or numbers of connections and/or configurations to other devices and/or elements can also be used.

By way of example only, the communication network(s) 22 can include local area network(s) (LAN(s)) or wide area network(s) (WAN(s)), and can use TCP/IP over Ethernet and industry-standard protocols, although other types and/or numbers of protocols and/or communication networks can be used. The communication network(s) 22 in this example can employ any suitable interface mechanisms and network communication technologies including, for example, teletraffic in any suitable form (e.g., voice, modem, and the like), Public Switched Telephone Network (PSTNs), Ethernet-based Packet Data Networks (PDNs), combinations thereof, and the like. The communication network(s) 22 can also include direct connection(s).

While the treatment management computing apparatus 12 is illustrated in this example as including a single device, the treatment management computing apparatus 12 in other examples can include a plurality of devices or blades each having one or more processors (each processor with one or more processing cores) that implement one or more steps of this technology. In these examples, one or more of the devices can have a dedicated communication interface or memory. Alternatively, one or more of the devices can utilize the memory, communication interface, or other hardware or software components of one or more other devices included in the treatment management computing apparatus 12.

Additionally, one or more of the devices that together comprise the treatment management computing apparatus 12 in other examples can be standalone devices or integrated with one or more other devices or apparatuses, such as one of the server devices, for example. Moreover, one or more of the devices that comprise the treatment management computing apparatus 12 in these examples can be in a same or a different communication network including one or more public, private, or cloud networks, for example.

The technology disclosed herein is not to be construed as being limited to a single environment and other configurations and architectures are also envisaged. For example, one or more of server devices can operate within the treatment management computing apparatus 12 itself rather than as a stand-alone server device communicating with the treatment management computing apparatus 12 via the communication network(s) 22. In this example, the one or more server devices operate within the memory of the network traffic management apparatus.

Each of the provider computing devices 14(1)-14(n) and each of the client computing devices 16(1)-16(n) may include a processor, a memory, and a communication interface, which are coupled together by a bus or other link, although other type and/or numbers of other devices and/or nodes as well as other network elements could be used. In this example, the provider computing devices 14(1)-14(n) may be operated by healthcare providers, such as a senior clinical manger, a clinical manager, and/or a clinical assessor by way of example only, and the client computing devices 16(1)-16(n) may be operated by the clients and/or associated entities, such as parents, relatives, or other caregivers by way of example only.

Each of the client sensor devices 17(1)-17(n) may include a processor, a memory, one or more sensing elements, and a communication interface, which are coupled together by a bus or other link, although other type and/or numbers of other devices and/or nodes as well as other network elements could be used. The one or more sensor devices 17(1)-17(n) may be configured and can be positioned to capture about biofeedback data about the client associated with the client identifier, such as heart rate, perspiration, and/or body temperature and/or more environmental factors, such as data on current temperature, pressure, and/or time of day, in a current environment for the client.

Each of the clinical server devices 18(1)-18(n) and each of the external data server devices 20(1)-20(n) may include a processor, a memory, and a communication interface, which are coupled together by a bus or other link, although other type and/or numbers of other devices and/or nodes as well as other network elements could be used. Each of the clinical server devices 18(1)-18(n) may store clinical data and other clinical standards and rules, although other types of information, data, or other executable instructions may be stored. Each of the external data server devices 20(1)-20(n) may store client specific data including situational data and preference data associated with client identifiers as well as past treatment data index or stored based on a type of condition or conditions being treated, although other types of data for generating the treatment plan data may be stored.

Although an exemplary environment with the treatment management computing apparatus 12, provider computing devices 14(1)-14(n), client computing devices 16(1)-16(n), client sensor devices 17(1)-17(n), clinical server devices 18(1)-18(n), and external data server devices 20(1)-20(n) are described and illustrated herein, other types and/or numbers of systems, devices, components, and/or elements in other topologies can be used. It is to be understood that the systems of the examples described herein are for exemplary purposes, as many variations of the specific hardware and software used to implement the examples are possible, as will be appreciated by those skilled in the relevant art(s).

One or more of the components depicted in the treatment management computing apparatus 12, for example, may be configured to operate as virtual instances on the same physical machine. In other words, one or more aspects of the treatment management computing apparatus 12 may operate on the same physical device rather than as separate devices communicating through communication network(s). Additionally, there may be more or fewer treatment management computing apparatus 12 than illustrated in FIG. 2.

In addition, two or more computing systems or devices can be substituted for any one of the systems or devices in any example. Accordingly, principles and advantages of distributed processing, such as redundancy and replication also can be implemented, as desired, to increase the robustness and performance of the devices and systems of the examples. The examples may also be implemented on computer system(s) that extend across any suitable network using any suitable interface mechanisms and traffic technologies, including by way of example only teletraffic in any suitable form (e.g., voice and modem), wireless traffic networks, cellular traffic networks, Packet Data Networks (PDNs), the Internet, intranets, and combinations thereof.

The examples may also be embodied as one or more non-transitory computer readable media having instructions stored thereon for one or more aspects of the present technology as described and illustrated by way of the examples herein. The instructions in some examples include executable code that, when executed by one or more processors, cause the processors to carry out steps necessary to implement the methods of the examples of this technology that are described and illustrated herein.

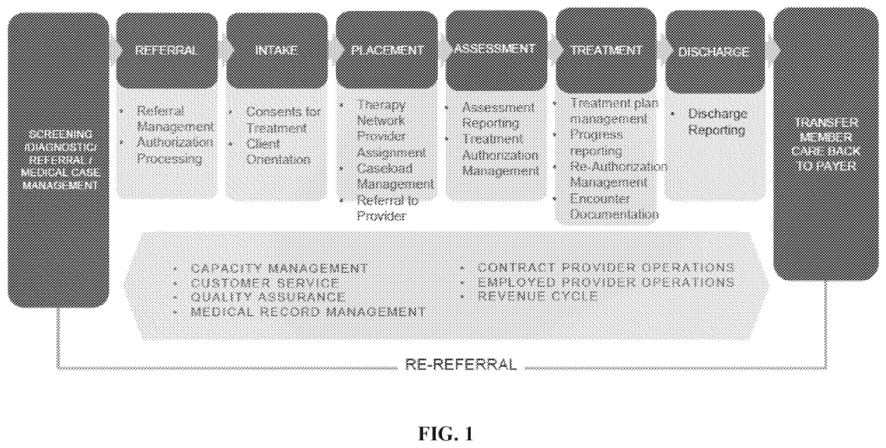

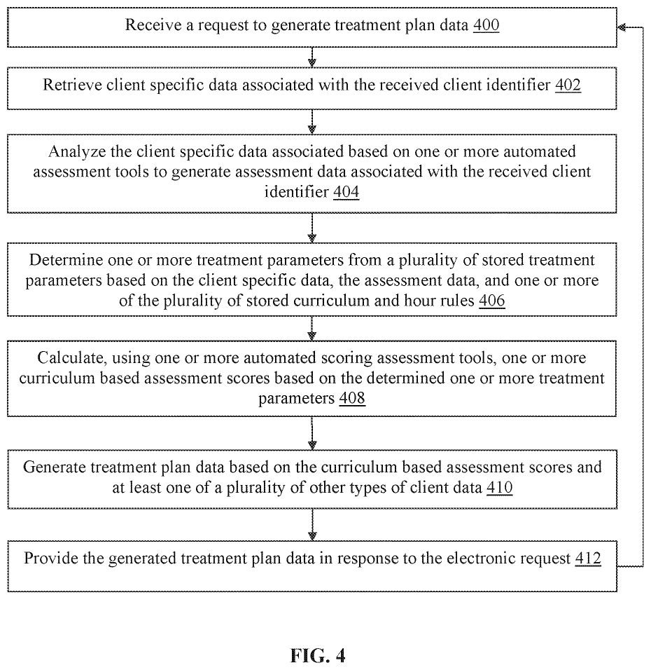

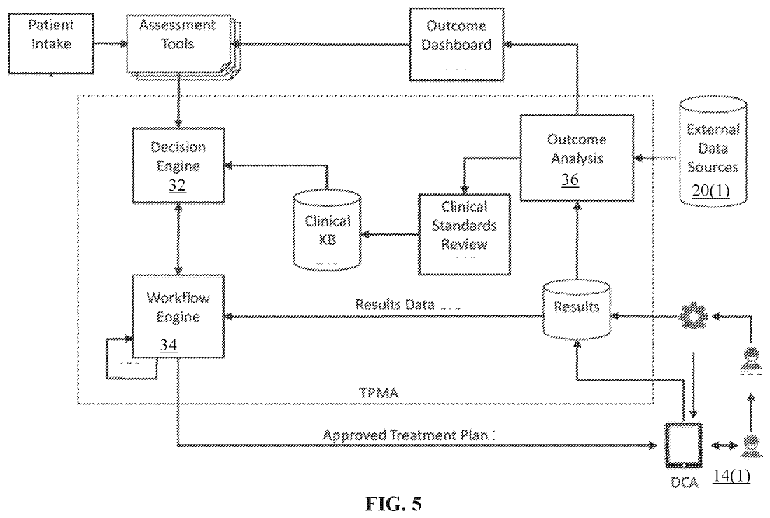

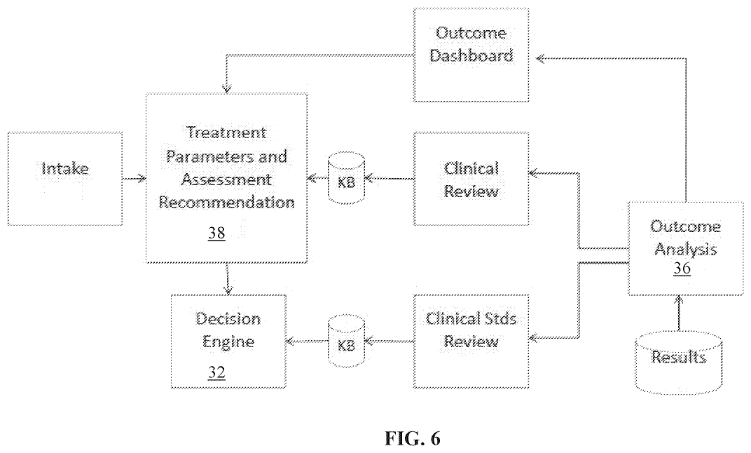

An example of a method for applying advanced multi-step analytics to generate treatment plan data associated with in this example a client identifier for a client with Autism Spectrum Disorder (ASD) will now be described with reference to FIGS. 1-5 and 7-9C. More specifically, a flow chart of this example of the method for applying advanced multi-step analytics to generate treatment plan data associated with a client identifier for a client with Autism Spectrum Disorder (ASD) is illustrated in FIG. 4 and two examples in FIGS. 5 and 6 of functional block diagrams of examples of how this flow chart can be implemented are illustrated. The example of the functional block diagram in FIG. 6 simplifies the flow from the example of the functional block diagram in FIG. 5.

Referring more specifically to FIG. 4, in step 400 the treatment management computing apparatus 12 may receive an electronic request or other communication from one of the provider computing devices 14(1)-14(n) operated by a mental healthcare provider to generate treatment plan data for a client identifier for a client with, in this example, an Autism Spectrum Disorder (ASD). In this example, the received request to generate the treatment plan data for the received client identifier may be a new request, although other types of requests can be received, such as a request to generate treatment plan data, which is an update for previously generated treatment plan data for the received client identifier if treatment is already in progress.

In step 402, the treatment management computing apparatus 12 may retrieve client specific data associated with the received client identifier from one of the external data servers 20(1)-20(n), one or more of the provider computing devices 14(1)-14(n), one or more of the client computing devices 16(1)-16(n), and/or one or more client sensor devices 17(1)-17(n), although the client specific data may be obtained from other sources. By way of example, the client specific data may include personal and medical information as well as situational data and preference data associated with the client identifier for the client as well as other related data for one or more caregivers for the client, such as one or more parents, relatives, and/or friends, and sensor data, such as biofeedback and/or environmental data by way of example only, although other types of data may be obtained and the data may be obtained from other types and/or numbers of sources. The situational data may comprise data associated with the client identifier for the client about stored reactions to particular situations and other environment factors which may impact or influence, although other types of situational data may be stored. Additionally, the preference data may comprise data associated with the client identifier for the client about particular preferences, such as data relating to particular places, people, and/or times of day, although other types of preference data may be stored. Additionally, as noted above the data may be obtained from other sources, such as inputs from one of the client computing devices 16(1)-16(n) associated with the client identifier or one or more of the provider computing devices 16(1)-16(n) by way of example only.

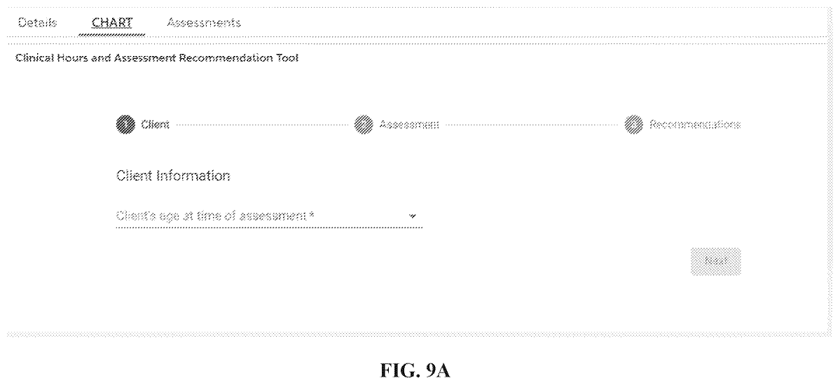

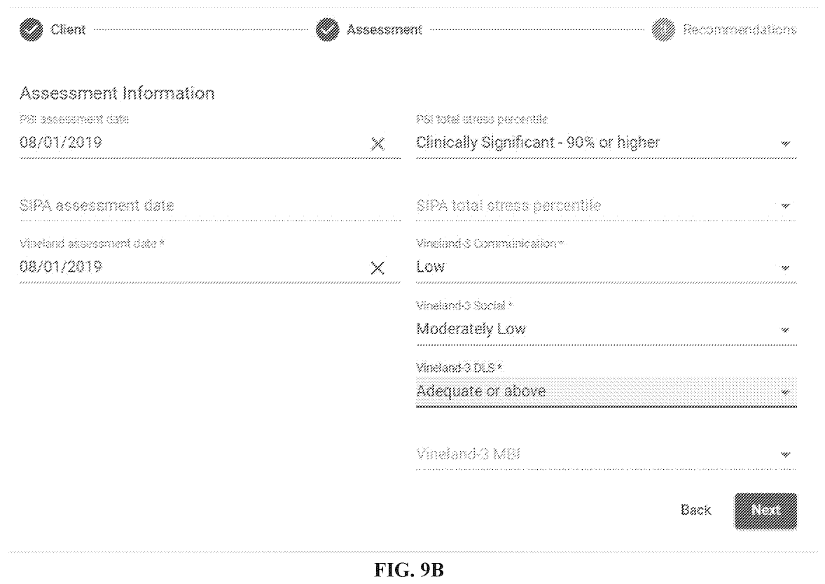

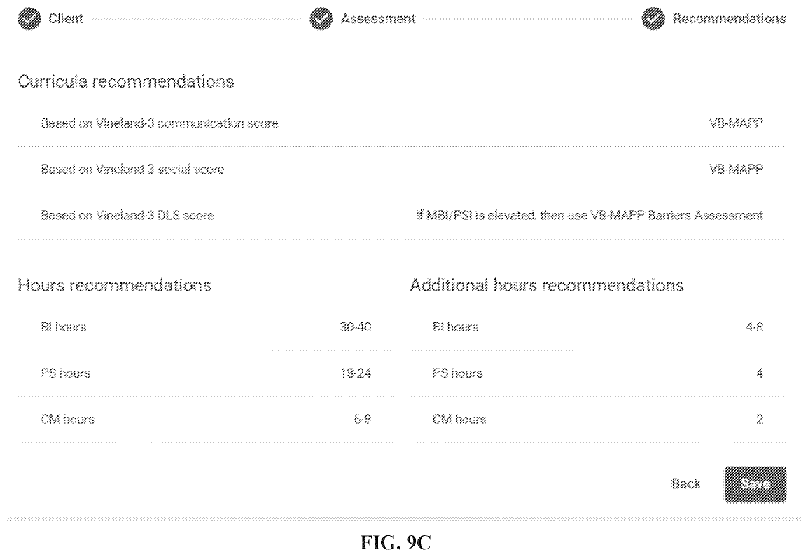

In step 404, the treatment management computing apparatus 12 may analyze the client specific data associated with a client identifier based on one or more automated assessment tools to generate assessment data associated with the client identifier. By way of example, the client specific data may be analyzed by the treatment management computing apparatus 12 based on an adaptive behavior age assessment, a parental stress assessment or parent stress index (PSI), or a parental stress assessment or stress index for parents of adolescents (SA) based on client specific data including an actual and/or a behavioral age of the client associated with the client identifier, although other types and/or numbers of assessment tools and/or other analytics may be used. By way of example only, a screenshot of an example of a graphical user interface of an example of a clinical hours and assessment recommendation tool provided by the treatment management computing apparatus 12 to one of the provider computing devices 14(1)-14(n) is illustrated in FIG. 9A.

In this particular example, the treatment management computing apparatus 12 may execute programmed instructions for a Vineland-3 assessment which is used to determine the adaptive behavior age equivalent of the client associated with the client identifier based on client specific data provide by one of the provider computing devices 14(1)-14(n) by way of example Additionally, the treatment management computing apparatus 12 may execute programmed instructions for the parental stress assessment or parent stress index (PSI) for the client identifier associated with an age below a set threshold, or a parental stress assessment or stress index for parents of adolescents (SA) based on an actual and/or a behavioral age of the client associated with the client identifier as well as other client specific data, such as data on stress levels or other factors of one or more parents or other caregivers of the client associated with the client identifier. The determined PSI or SIPA may be used in examples of this technology by the treatment management computing apparatus 12 to determine need for additional supervision hours for education or support for the one or more parents or other caregivers of the client associated with the client identifier in the generated treatment plan data.

A table illustrating these examples of the assessment tools is illustrated below:

| Step 1 Assessment Tool Name | Referencing | Age | |
|---|---|---|---|
| Vineland Adaptive Behavior | Norm | 0-90 | years |
| Scales - Third Edition | | | |
| (Vineland-3) | | | |
| Parenting Stress Index - 4 | Norm | 0-12 | years |
| Stress Index for Parents | Norm | 11-19 | years |
| of Adolescents (SIPA) | | | |

By way of example, the Vineland-3 assessment may include one or more bulk domain scores, including scores from the Communications, Socialization, Daily Living Skills, and Maladaptive domains. For each domain, these scores can be used to determine a client's developmental levels (e.g., Low, Moderate Low, or Adequate) to be used for generating the treatment plan data. In another example, finer granularity by the treatment management computing apparatus 12 may be achieved by processing data related to subdomains of the Vineland-3 assessment. The Vineland-3 results could be evaluated by subdomains by the treatment management computing apparatus 12, as well, which could lead to tracking of the number of treatment goals recommended per subdomain level by the treatment management computing apparatus 12. In addition to this, the PSI and SIPA assessment could also be evaluated by subdomains by the treatment management computing apparatus 12, specifically notating client specific data on what are parental areas of stress that could be targeted during treatment, or would be better addressed by outside referral to automatically adjust the generated treatment plan data.

By way of example, a screen shot of an example a customized graphical user interface (GUI) of an example of the technology that may be used by a provider at one of the provider computing devices 14(1)-14(n) during operation of an example of this technology is illustrated in FIG. 9.

Referring back to FIG. 4, in step 406, the treatment management computing apparatus 12 may determine one or more treatment parameters from a plurality of stored treatment parameters based on the client specific data, the assessment data, and one or more of the plurality of stored curriculum and hour rules. The treatment management computing device 12 may correlate the generated assessment data and/or other data to a table of a plurality of treatment curriculum and hours rules as illustrated in FIG. 7 and/or to an additional hours table as illustrated in FIG. 8 to determine one or more treatment parameters, although other manners for determining one or more treatment parameters may be used. By way of another example, the treatment management computing apparatus 12 may use client specific data and/or the generated assessment data to identify one or more curriculum and associated ranges of hours that may be appropriate for the client associated with the client identifier from one or more external data server devices 20(1)-20(n) which may store current best practices and/or research for addressing one or more types of a plurality of diagnosed conditions. As a result, examples of the claimed technology are able to utilize the most recent best practices and research and this utilization between providers at one or more of the provider computing devices 14(1)-14(n) is standardized and thus more consistent. By way of example only, a screenshot of an example of a graphical user interface of generated assessment data provided by the treatment management computing apparatus 12 to a requesting one of the provider computing devices 14(1)-14(n) is illustrated in FIG. 9B.

In step 408, the treatment management computing apparatus 12 may calculate one or more curriculum based assessment scores using one or more automated scoring assessment tools based on the determined one or more treatment parameters to determine one or more curriculum based assessment scores. In this particular example to calculate the one or more treatment scores, the treatment management computing apparatus 12 may execute programmed instructions to analyze the determined treatment parameters as well as client specific data associated with a client identifier to calculate one or more curriculum based assessment scores. By way of example, the treatment management computing apparatus 12 may calculate the determined curriculum based assessment scores based on programmed instructions for a verbal behavior milestones assessment and placement program tool (VB-MAPP), an assessment of functional living skills tool (AFLS), an essential for living tool (EFL), and/or a behavior rating inventory of executive function tool (BREIF2) although other types and/or numbers of scoring assessment tools and/or other analytics may be used

A table illustrating these examples of the scoring assessment tools is illustrated below:

| Curriculum/Step 2 | | |
|---|---|---|
| Assessment Tool Name | Referencing | Age |
| Verbal Behavior | Criterion | 0-48 months |
| Milestones Assessment and | | |
| Placement Program (VB-MAPP) | | |
| The Assessment of Functional | Criterion | 2 years and up |
| Living Skills (AFLS) | | |
| Essential for Living (EFL) | Criterion | Children and Adults |
| Behavior Rating Inventory | Norm | 5-18 Parent/ |
| of Executive Function | | Teach form |
| (BRIEF2) | | 11-18 Self- |
| | | Report Form |

In step 410, the treatment management computing apparatus 12 may generate treatment plan data based on one or more stored rules associated with the determined one of a plurality of diagnosed conditions associated with the client identifier (for example obtained by analyzing the client specific data or otherwise input), the one or more determined treatment parameters, and the one or more curriculum based assessment scores. By way of example, if the determined treatment parameters provided an ‘Hour Recommendation Range’ for a 2-year-old client between 20-40 hours, and results of the assessment tool for (VB-MAPP) reveal challenges in skill acquisition/generalization (for the VBMAPP, this would include scoring low in level 1 and identified barriers to progress), the treatment management computing apparatus 12 may generated treatment plan data for direct service hours on the higher end of the stored range correlated with current best practices from other correlated stored data for other clients with matching diagnosed conditions.

By way of example, if the results of the assessment tool provided to the treatment management computing device 12 indicate a treatment score for higher skill acquisition (e.g. the client scoring high level 2/level 3 on VBMAPP, with fewer identified barriers to progress), the treatment management computing device 12 may generated treatment plan data for direct service hours on the lower end of the stored range correlated with current best practices from other correlated stored data for other clients with matching diagnosed conditions

In another example, the treatment management computing device 12 may execute artificial intelligence (AI) trained on prior stored client outcome data and correlated to the client specific data and the assessment data to generate executable rules for adjusting one or more of the curriculum based assessment scores calculated using the one or more automated scoring assessment tools be implemented to leverage AI, in order to assist in managing the different rules that are used to recommend assessment curriculum and treatment parameters.

By way of example, a table with curriculum based assessment scores and generated treatment plan data is illustrated below:

| | | Output | |
|---|---|---|---|
| Input | | Curriculum | Hours |
| Age | 4 y | VB-MAPP | Baseline: BI: 20/ |
| Develop- | Low (Comm = Low, | | PS: 18/CM: 5 |
| mental | Social = Low, | | |
| Level | DLS = Low) | | |
| PSI | High Range | | Additional: BI: 0/ |
| MBI | Elevated | | PS: 2/CM: 1 |
| | | | Total Hours: BI: 20/ |
| | | | PS: 20/CM: 6 |

Additionally by way of example only, a screenshot of an example of a graphical user interface of treatment plan data comprising curricula recommendations, hours recommendations, and additional hours recommendations provided by the treatment management computing apparatus 12 to a requesting one of the provider computing devices 14(1)-14(n) is illustrated in FIG. 9C, although other types of treatment plan data may be provided.

In step 412, the treatment management computing apparatus 12 may provide the generated treatment plan data is transmitted in response to the electronic request back to for example one of the provider computing devices 14(1)-14(n) and/or one of the client computing devices 16(1)-16(n). This example of the method may end here or in other examples may return to step 400 when an update of the generated treatment plan data is needed.

Accordingly, as illustrated and described by way of the examples herein, this technology provides providing methods, devices, and non-transitory computer readable media that apply advanced multi-step analytics to generate treatment plan data that is aligned with current best practices and research and effectively and consistently tailored to each client. The advanced multi-step analytics utilize client specific data including situational data and preference data for a client as well as other stored client outcome data correlated to other client identifiers with one or more matching types of diagnosis conditions. Additionally, these advanced multi-step analytics may utilize artificial intelligence (AI) to analyze data including the client specific data and the other stored client outcome data including stored treatment outcome data in order to generate and continually update executable rules for new assessments and adjustments to treatment parameters for generating mental healthcare treatment plan data. Further, these advanced multi-step analytics may include analytics to customize the duration of different parts of the treatment strategy. Accordingly, this technology generates much more effective and consistent treatment plan data and does so in substantially less time. Further, with this technology any changes to the particular healthcare team or between different healthcare providers associated with each client identifier no longer impacts the generated treatment plan data for each client identifier.

Having thus described the basic concept of the invention, it will be rather apparent to those skilled in the art that the foregoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and scope of the invention. Additionally, the recited order of processing elements or sequences, or the use of numbers, letters, or other designations therefore, is not intended to limit the claimed processes to any order except as may be specified in the claims. Accordingly, the invention is limited only by the following claims and equivalents thereto.

What is claimed is:

1. A method for applying advanced multi-step analytics to generate plan data with improved data processing efficiency, the method comprising:
receiving, by a processor of a computing device via a communication interface, an electronic request from a provider computing device over a communication network;
retrieving, by the processor, client specific data associated with a client identifier from one or more external data server devices over the communication network;
analyzing, by the processor, the client specific data based on one or more automated evaluation assessment tools stored in a memory coupled to the processor to generate evaluation data associated with the client identifier;
executing, by the processor, a decision engine stored in the memory to correlate the client specific data with prior stored client outcome data retrieved from the one or more external data server devices to identify matching data patterns, thereby improving accuracy of data analysis;
querying, by the processor, one or more clinical server devices over the communication network to obtain one or more of a plurality of stored curriculum and hour rules;
executing, by the processor, a workflow engine stored in the memory to automatically process and correlate the evaluation data with the obtained one or more of the plurality of stored curriculum and hour rules to determine one or more parameters from a plurality of stored parameters without manual intervention, thereby reducing processing time;
calculating, by the processor, using one or more automated scoring assessment tools stored in the memory one or more plan data scores based on the determined one or more parameters to determine one or more data scores;
generating, by the processor, plan data by automatically correlating the plan data scores with at least one of a plurality of other types of client data stored in the memory, thereby reducing processing time compared to manual data correlation;
storing, by the processor, the generated plan data in the memory; and
transmitting, by the processor via the communication interface, the generated plan data over the communication network to the provider computing device in response to the electronic request, wherein the transmitting standardizes data processing across multiple provider computing devices connected to the communication network.

2. The method as set forth in claim 1 wherein the one or more automated evaluation assessment tools comprise one or more of an adaptive behavior age assessment, a parental stress assessment for a client identifier associated with an age below a set threshold, or a parental stress assessment for a client identifier associated with an age at or above the set threshold.

3. The method as set forth in claim 1 wherein the one or more parameters comprise one or more of a plurality of curriculum and a range of hours for each of the plurality of curriculum stored in a curriculum rules database in the memory.

4. The method as set forth in claim 1 wherein the one or more automated scoring assessment tools comprise one or more of a verbal behavior milestones assessment and placement program tool, an assessment of functional living skills tool, an essential for living tool, or a behavior rating inventory of executive function tool.

5. The method as set forth in claim 1 further comprising:
automatically retrieving, by the processor via the communication interface, sensor data from a plurality of client sensor devices over the communication network, wherein the sensor data comprises at least one of biofeedback data or environmental data associated with the client identifier;
correlating, by the processor, the sensor data from the plurality of client sensor devices to identify data patterns; and
storing, by the processor, the sensor data in the memory, wherein the other types of client data comprises the sensor data, situational data, and preference data associated with the client identifier.

6. The method as set forth in claim 1 wherein the calculating using the one or more automated scoring assessment tools further comprises:
executing, by the processor, an artificial intelligence engine stored in the memory, the artificial intelligence engine trained on prior stored client outcome data correlated to the client specific data and the evaluation data to generate executable rules stored in the memory for adjusting one or more plan data scores calculated using the one or more automated scoring assessment tools, wherein the artificial intelligence engine analyzes patterns in the prior stored client outcome data correlated to matching diagnosed conditions, generates and updates predictive models stored in the memory based on the analyzed patterns, and continuously refines the executable rules by comparing predicted outcomes with actual outcomes received from the one or more external data server devices, wherein the iterative learning process improves accuracy of the one or more plan data scores over successive iterations, wherein the artificial intelligence engine weights data patterns stored in the memory based on outcome success rates retrieved from the one or more external data server devices, adjusts scoring parameters stored in the memory based on correlation strength between client specific data patterns and successful outcomes, and updates weighting factors in the memory based on statistical analysis of outcome data, resulting in progressively more accurate plan data generation over successive processing cycles.

7. The method as set forth in claim 1 wherein the generating the plan data based on the plan data scores further comprises:
querying, by the processor, an hours rules database stored in the memory to obtain one or more determined ranges; and
determining, by the processor, a time duration of one or more parts of the plan data based on the one or more determined ranges from the hours rules database.

8. A non-transitory computer readable medium having stored thereon instructions comprising executable code which when executed by one or more processors coupled to a memory and a communication interface, causes the one or more processors to:
receive, via the communication interface, an electronic request from a provider computing device over a communication network;
retrieve client specific data associated with a client identifier from one or more external data server devices over the communication network;
analyze the client specific data based on one or more automated evaluation assessment tools stored in the memory to generate evaluation data associated with the client identifier;
execute a decision engine stored in the memory to correlate the client specific data with prior stored client outcome data retrieved from the one or more external data server devices to identify matching data patterns, thereby improving accuracy of data analysis;
query one or more clinical server devices over the communication network to obtain one or more of a plurality of stored curriculum and hour rules;
execute a workflow engine stored in the memory to automatically process and correlate the evaluation data with the obtained one or more of the plurality of stored curriculum and hour rules to determine one or more parameters from a plurality of stored parameters without manual intervention, thereby reducing processing time;
calculate using one or more automated scoring assessment tools stored in the memory one or more plan data scores based on the determined one or more parameters to determine one or more data scores;
generate plan data by automatically correlating the plan data scores with at least one of a plurality of other types of client data stored in the memory, thereby reducing processing time compared to manual data correlation;
store the generated plan data in the memory; and
transmit, via the communication interface, the generated plan data over the communication network to the provider computing device in response to the electronic request, wherein the transmitting standardizes data processing across multiple provider computing devices connected to the communication network.

9. The medium as set forth in claim 8 wherein the one or more automated evaluation assessment tools comprise one or more of an adaptive behavior age assessment, a parental stress assessment for a client identifier associated with an age below a set threshold, or a parental stress assessment for a client identifier associated with an age at or above the set threshold.

10. The medium as set forth in claim 8 wherein the one or more parameters comprise one or more of a plurality of curriculum and a range of hours for each of the plurality of curriculum stored in a curriculum rules database in the memory.

11. The medium as set forth in claim 8 wherein the one or more automated scoring assessment tools comprise one or more of a verbal behavior milestones assessment and placement program tool, an assessment of functional living skills tool, an essential for living tool, or a behavior rating inventory of executive function tool.

12. The medium as set forth in claim 8 wherein the executable code when executed by the one or more processors further causes the one or more processors to:
automatically retrieve, via the communication interface, sensor data from a plurality of client sensor devices over the communication network, wherein the sensor data comprises at least one of biofeedback data or environmental data associated with the client identifier;
correlate the sensor data from the plurality of client sensor devices to identify data patterns; and
store the sensor data in the memory, wherein the other types of client data comprises the sensor data, situational data, and preference data associated with the client identifier.

13. The medium as set forth in claim 8 wherein for the calculate using the one or more automated scoring assessment tools, the executable code when executed by the one or more processors further causes the one or more processors to:
execute an artificial intelligence engine stored in the memory, the artificial intelligence engine trained on prior stored client outcome data correlated to the client specific data and the evaluation data to generate executable rules stored in the memory for adjusting one or more plan data scores calculated using the one or more automated scoring assessment tools, wherein the artificial intelligence engine analyzes patterns in the prior stored client outcome data correlated to matching diagnosed conditions, generates and updates predictive models stored in the memory based on the analyzed patterns, and continuously refines the executable rules by comparing predicted outcomes with actual outcomes received from the one or more external data server devices, wherein the iterative learning process improves accuracy of the one or more plan data scores over successive on outcome success rates retrieved from the one or more external data server devices, adjusts scoring parameters stored in the memory based on correlation strength between client specific data patterns and successful outcomes, and updates weighting factors in the memory based on statistical analysis of outcome data, resulting in progressively more accurate plan data generation over successive processing cycles.

14. The medium as set forth in claim 8 wherein for the generate the plan data based on the plan data scores, the executable code when executed by the one or more processors further causes the one or more processors to:
query an hours rules database stored in the memory to obtain one or more determined ranges; and
determine a time duration of one or more parts of the plan data based on the one or more determined ranges from the hours rules database.

15. A computing apparatus comprising:
a processor;
a communication interface coupled to the processor via a bus and configured to communicate over a communication network with a provider computing device, one or more external data server devices, and one or more clinical server devices;
a memory coupled to the processor via the bus, the memory storing a decision engine, a workflow engine, an outcome analysis tool, a parameter recommendation tool, a curriculum rules database, and an hours rules database; and
wherein the processor is configured to execute programmed instructions stored in the memory to:
receive, via the communication interface, an electronic request from the provider computing device over the communication network;
retrieve client specific data associated with a client identifier from the one or more external data server devices over the communication network;
analyze, using the decision engine, the client specific data based on one or more automated evaluation assessment tools to generate evaluation data associated with the client identifier;
execute the decision engine to correlate the client specific data with prior stored client outcome data retrieved from the one or more external data server devices to identify matching data patterns, thereby improving accuracy of data analysis;
query the one or more clinical server devices over the communication network to obtain one or more of a plurality of stored curriculum and hour rules;
execute the workflow engine to automatically process and correlate the evaluation data with the obtained one or more of the plurality of stored curriculum and hour rules to determine, using the parameter recommendation tool, one or more parameters from a plurality of stored parameters without manual intervention, thereby reducing processing time;
calculate, using the workflow engine, one or more plan data scores based on the determined one or more parameters using one or more automated scoring assessment tools to determine one or more data scores;
generate plan data by automatically correlating the plan data scores with at least one of a plurality of other types of client data stored in the memory, thereby reducing processing time compared to manual data correlation;
store the generated plan data in the memory; and
transmit, via the communication interface, the generated plan data over the communication network to the provider computing device in response to the electronic request, wherein the transmitting standardizes data processing across multiple provider computing devices connected to the communication network.

16. The apparatus as set forth in claim 15 wherein the one or more automated evaluation assessment tools comprise one or more of an adaptive behavior age assessment, a parental stress assessment for a client identifier associated with an age below a set threshold, or a parental stress assessment for a client identifier associated with an age at or above the set threshold.

17. The apparatus as set forth in claim 15 wherein the one or more parameters comprise one or more of a plurality of curriculum and a range of hours for each of the plurality of curriculum stored in the curriculum rules database.

18. The apparatus as set forth in claim 15 wherein the one or more automated scoring assessment tools comprise one or more of a verbal behavior milestones assessment and placement program tool, an assessment of functional living skills tool, an essential for living tool, or a behavior rating inventory of executive function tool.

19. The apparatus as set forth in claim 15 wherein the processor is further configured to execute programmed instructions stored in the memory to:
automatically retrieve, via the communication interface, sensor data from a plurality of client sensor devices over the communication network, wherein the sensor data comprises at least one of biofeedback data or environmental data associated with the client identifier;
correlate the sensor data from the plurality of client sensor devices to identify data patterns; and
store the sensor data in the memory, wherein the other types of client data comprises the sensor data, situational data, and preference data associated with the client identifier.

20. The apparatus as set forth in claim 15 wherein for the calculate using the one or more automated scoring assessment tools, the processor is further configured to execute programmed instructions stored in the memory to:
execute an artificial intelligence engine stored in the memory, the artificial intelligence engine trained on prior stored client outcome data correlated to the client specific data and the evaluation data to generate executable rules stored in the memory for adjusting one or more plan data scores calculated using the one or more automated scoring assessment tools, wherein the artificial intelligence engine analyzes patterns in the prior stored client outcome data correlated to matching diagnosed conditions, generates and updates predictive models stored in the memory based on the analyzed patterns, and continuously refines the executable rules by comparing predicted outcomes with actual outcomes received from the one or more external data server devices, wherein the iterative learning process improves accuracy of the one or more plan data scores over successive on outcome success rates retrieved from the one or more external data server devices, adjusts scoring parameters stored in the memory based on correlation strength between client specific data patterns and successful outcomes, and updates weighting factors in the memory based on statistical analysis of outcome data, resulting in progressively more accurate plan data generation over successive processing cycles.

21. The apparatus as set forth in claim 15 wherein for the generate the plan data based on the plan data scores, the processor is further configured to execute programmed instructions stored in the memory to:
query the hours rules database stored in the memory to obtain one or more determined ranges; and
determine a time duration of one or more parts of the plan data based on the one or more determined ranges from the hours rules database.
